SEQUENCE LISTING (3 sequences)

SEQ ID NO: 1 - DNA, 20 bp, Artificial Sequence
  The sequence is synthetized.
catcgagtacatccgcgaac

SEQ ID NO: 2 - DNA, 20 bp, Artificial Sequence
  The sequence is synthetized.
aaatcagcaatcagcgcgtc

SEQ ID NO: 3 - DNA, 132 bp, Unknown
  cable bacteria (Candidatus Electronema)
aaatcagcaatcagcgcgtcgcatgactctttgcagtcagtcatgaactccacgttgttgcgagtggtgaagcgcagatagcccttgcagtatttgtcagcgatgtcgcaaagttcgcggatgtactcgatg

(19) United States
(12) Patent Application Publication
(10) Pub. No.: US 2021/0285029 A1
(43) Pub. Date: Sep. 16, 2021

(54) PRIMERS, KITS AND METHODS FOR THE DETECTION AND QUANTITATION OF CABLE BACTERIA (CANDIDATUS ELECTRONEMA)

(71) Applicant: Guangdong Institute of Microbiology (Guangdong Detection Center of Microbiology), Guangzhou (CN)

(72) Inventors: Meiying XU, Guangzhou (CN); Wenzhe HU, Guangzhou (CN); Bin WANG, Guangzhou (CN); Da SONG, Guangzhou (CN); Jun GUO, Guangzhou (CN)

(73) Assignee: Guangdong Institute of Microbiology (Guangdong Detection Center of Microbiology), Guangzhou (CN)

(21) Appl. No.: 17/257,319

(22) PCT Filed: Sep. 17, 2019
(86) PCT No.: PCT/CN2019/106080

§ 371 Date: Dec. 31, 2020

(30) Foreign Application Priority Data
Jun. 25, 2019 (CN) 201910555540.9

(51) Int. Cl. C12Q 1/689; C12Q 1/6851

(52) U.S. Cl. CPC C12Q 1/689; C12Q 2600/166; C12Q 1/6851

(57) ABSTRACT

Disclosed are primers, kits and methods for the detection and quantitation of cable bacteria (Candidatus Electronema). Use of the primers by the methods enables the detection and quantitation of cable bacteria (Candidatus Electronema) in environmental samples, with a minimum detection limit of 10 copies/μL, resulting in the sensitivity 10,000 times higher than that of the currently used FISH method. The primers, the kit, and the methods have high sensitivity, high accuracy, good reproducibility, and high specificity, and allow detection with a linear range of 10¹-10⁸ copies/μL.

### CROSS REFERENCE TO THE RELATED APPLICATIONS

This application is the national phase entry of International Application No. PCT/CN2019/106080, filed on Sep. 17, 2019, which is based upon and claims priority to Chinese Patent Application No. 201910555540.9, filed on Jun. 25, 2019, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBKY062_sequence_listing_ST25.txt and is 1,181 bytes in size.

### TECHNICAL FIELD

The present invention relates to the technical field of molecular biology, and particularly relates to primers, kits and methods for the detection and quantitation of cable bacteria (Candidatus Electronema).

### BACKGROUND

Cable bacteria, a type of long filamentous multicellular bacteria in the family Desulfobulbaceae, can generate electrical currents across centimeter-scale distances through long-distance electron transfer (LDET) linking sulfide oxidation in deeper anoxic zones to oxygen reduction in surface oxic zones which are spatially segregated in sediments. This finding challenges the long-accepted view that redox zonation is controlled by molecule diffusion. Cable bacteria are widely distributed around the world and have been found in various sediment environments such as marine sediments, mangrove sediments, salt marsh sediments, and freshwater sediments, playing a significant role in driving the global biogeochemical cycle. When the LDET mediated by cable bacteria is active, cable bacteria can promote the dissolution of iron sulfides, inducing the transposition and redistribution of dissolved sulphur, iron, and calcium in the sediments, coupling the Fe—P and Fe—Mn cycles in the sediments and bottom water, and forming dense iron oxyhydroxide or iron oxide over the sediment surface to prevent the toxic hydrogen sulfide in the sediments from diffusing into the water, which thereby protects organisms living in the water. The cable bacteria are currently a very exciting discovery in the electromicrobiology, which have attracted more and more research and attention from domestic and foreign researchers.

However, the pure culture of cable bacteria cannot be obtained so far. Although the fluorescence in situ hybridization (FISH) combing with the line intersection method has been used in cable bacteria quantification, the sensitivity with a minimum quantitative detection limit of 1.5*10⁶ cells per cubic centimeter is too low to satisfy the requirements. The fluorescence quantitative PCR method has become the preferred molecular biology method for the quantitative detection of low-abundance species in the environment for its high specificity, high sensitivity, accurate quantitation, good reproducibility, and high speed. Thus, how to establish a fluorescence quantitative PCR protocol for quantitative detection of cable bacteria in the environment has been one of the technical challenges.

### SUMMARY

In view of the poor sensitivity and accuracy of quantitation of cable bacteria in the environment using prior art technology, one object of the present invention is to provide primers, kits and methods for the detection and quantitation of cable bacteria (Candidatus Electronema), which allow for rapid quantitative detection of cable bacteria in the environment and improve the detection sensitivity.

A first object of the present invention is to provide primers for detecting cable bacteria (Candidatus Electronema), comprising:

a forward primer:
(SEQ ID NO: 1)
5′-CATCGAGTACATCCGCGAAC-3′,
and
a reverse primer:
(SEQ ID NO: 2)
5′-AAATCAGCAATCAGCGCGTC-3′.

A second object of the present invention is to provide a kit for detecting cable bacteria (Candidatus Electronema), comprising a reagent necessary for qPCR detection (such as TB Green® Premix Ex Taq™ II), a positive control, and primers, wherein the positive control is a recombinant plasmid DNA comprising a sequence of SEQ ID NO: 3, and the primers are the primers of the first object.

A third object of the present invention is to provide a method for detecting cable bacteria (Candidatus Electronema), comprising the following steps: extracting genomic DNA from a sediment sample, mixing the genomic DNA with the primers and a reagent necessary for detection (such as TB Green® Premix Ex Taq™ II) to give an amplification reaction mixture, performing fluorescence quantitative PCR, and determining whether cable bacteria (Candidatus Electronema) are present in the sediment sample based on amplification curves.

The step of determining whether cable bacteria (Candidatus Electronema) are present in the sediment sample based on the amplification curves is performed according to the following standard: if the amplification curves comprise typical amplification curves and a cycle threshold (CT) value is below 35, then cable bacteria (Candidatus Electronema) are present in the sediment sample; if the amplification curves do not comprise the typical amplification curves, then cable bacteria (Candidatus Electronema) are not present in the sediment sample.

Preferably, the amplification reaction mixture comprises, per 25 μL of the amplification reaction mixture: 1 μL of a 10 μmol/L solution of the forward primer, 1 μL of a 10 μmol/L solution of the reverse primer, 12.5 μL of the reagent, 1 μL of an extracted solution of the genomic DNA, and 9.5 μL of ultrapure water. Also preferably, the step of performing fluorescence quantitative PCR comprises: (i) an initial denaturation step at 95° C. for 3 minutes, and (II) 40 cycles, wherein each of the cycles comprises a denaturation step at 95° C. for 5 seconds, an annealing step at 56° C. for 30 seconds, an extension step at 72° C. for 5 seconds, and a step of collecting fluorescent intensity.

A fourth object of the present invention is to provide a method for quantitating an abundance of cable bacteria (Candidatus Electronema), comprising the following steps:

(1) producing a recombinant plasmid by joining a sequence of SEQ ID NO: 3 with a plasmid, serially diluting the recombinant plasmid to give template solutions of a plurality of initial concentrations, mixing each of the template solutions with the primers and a reagent necessary for detection (such as TB Green® Premix Ex Taq™ II) to give an amplification reaction mixture, and performing fluorescence quantitative PCR;

(2) recording CT values corresponding to the initial concentrations, and obtaining a standard curve by plotting the CT values against common logarithms of the initial concentrations, wherein the CT values exhibit a linear relationship with the common logarithms of the initial concentrations;

(3) extracting genomic DNA from a sediment sample, mixing the genomic DNA with the primers and the reagent used in step (1) to give an amplification reaction mixture, performing fluorescence quantitative PCR, recording a sample CT value, and substituting the sample CT value into the standard curve to obtain the abundance of cable bacteria (Candidatus Electronema) in the sediment sample.

Preferably, in step (1) and step (3), the amplification reaction mixture comprises, per 25 μL of the amplification reaction mixture: 1 μL of a 10 μmol/L solution of the forward primer, 1 μL of a 10 μmol/L solution of the reverse primer, 12.5 μL of the reagent, 1 μL of an extracted solution of the genomic DNA, and 9.5 μL of ultrapure water. Also preferably, in step (1) and step (3), the step of performing fluorescence quantitative PCR comprises: (i) an initial denaturation step at 95° C. for 3 minutes, and (II) 40 cycles, wherein each of the cycles comprises a denaturation step at 95° C. for 5 seconds, an annealing step at 56° C. for 30 seconds, an extension step at 72° C. for 5 seconds, and a step of collecting fluorescent intensity.

Experimental results show that, use of the primers of the present invention by the provided methods enables the detection and quantitation of cable bacteria (Candidatus Electronema) in environmental samples, with a minimum detection limit of 10 copies/μL, resulting in the sensitivity 10,000 times higher than that of the currently used FISH method. The primers, the kit, and the methods have high sensitivity, high accuracy, good reproducibility, and high specificity, and allow detection with a linear range of 10¹-10⁸ copies/μL.

### BRIEF DESCRIPTION OF THE DRAWINGS

FIG. 1 shows the result of primer specificity test by electrophoresis in 1.2% agarose gel, wherein a 132 bp band is present, and M represents DL2000 Marker.

FIG. 2 shows the dissociation curve analysis.

FIG. 3 shows the PCR amplification kinetic curves, wherein 1 represents 10⁸ copies/μL, 2 represents 10⁷ copies/μL, 3 represents 10⁶ copies/μL, 4 represents 10⁵ copies/μL, 5 represents 10⁴ copies/μL, 6 represents 10³ copies/μL, 7 represents 10² copies/μL, and 8 represents 10¹ copies/μL.

FIG. 4 shows the PCR standard curve, wherein 1 represents 10¹ copies/μL, 2 represents 10² copies/μL, 3 represents 10³ copies/μL, 4 represents 10⁴ copies/μL, 5 represents 10⁵ copies/μL, 6 represents 10⁶ copies/μL, 7 represents 10⁷ copies/μL, and 8 represents 10⁸ copies/μL. The expression E=95.0% represents the amplification efficiency of the primers, and the expression R²=0.998 represents the linear correlation coefficient.

FIG. 5 shows the results of PCR detection, wherein 1 and 2 represent cable bacteria-rich positive samples, 3 represents a Desulfobulbus spp. of the family Desulfobulbaceae as a negative control, 4 represents E. coli as a negative control, and 5 represents water as a blank control.

FIG. 6 is an SEM (scanning electron microscope) image of a cable bacteria enrichment culture.

FIG. 7 shows the abundance of cable bacteria in the environmental samples determined by 16S rRNA gene sequence analysis.

FIG. 8 shows the abundance of cable bacteria in the environmental samples determined by fluorescence quantitative PCR.

### DETAILED DESCRIPTION OF THE EMBODIMENTS

The following description is to further illustrate the present invention, but not to limit the scope of the present invention.

1. The kit and methods of the present invention have high sensitivity, high accuracy, and good reproducibility, and allow detection with a linear range of 10¹-10⁸ copies/μL.

2. Specificity: The specific primers were designed using the sulfite reductase β subunit-encoding gene DsrB of cable bacteria (Candidatus Electronema) from the NCBI database. It has been validated that the kit allows quantitating the abundance of cable bacterial (Candidatus Electronema), but is negative to other species of the family Desulfobulbaceae.

### Embodiment 1

1) Designing Primers for Fluorescence Quantitative PCR

Cable bacteria are a type of long filamentous multicellular bacteria in the family Desulfobulbaceae. All sulfite reductase β subunit-encoding gene (DsrB) sequences of the family Desulfobulbaceae (downloaded from the GenBank database), and another DsrB gene sequence of a cable bacteria (Candidatus Electronema) strain from our team, were used to design specific primers of the DsrB gene sequence of cable bacteria (Candidatus Electronema) using Primer-BLAST. The expected amplicon size was 132 bp. The designed forward and reverse primers were as follows:

Forward primer:
(SEQ ID NO: 1)
5′-CATCGAGTACATCCGCGAAC-3′;
reverse primer:
(SEQ ID NO: 2)
5′-AAATCAGCAATCAGCGCGTC-3′.

2) Establishing PCR Amplification Protocol and Verifying PCR Products

DNA was extracted from cable bacteria-containing sediment samples using conventional methods and used as a template for PCR. The amplification reaction mixture comprises, per 25 μL of the amplification reaction mixture: 1 μL of a 10 μmol/L solution of the forward primer, 1 μL of a 10 μmol/L solution of the reverse primer, 12.5 μL of TB Green® Premix Ex Taq™ II, 1 μL of a solution of the extracted DNA (template DNA) from the sediment samples, and 9.5 μL of ultrapure water. The fluorescence quantitative PCR protocol comprised: (i) an initial denaturation step at 95° C. for 3 minutes, and (II) 40 cycles, wherein each of the cycles comprises a denaturation step at 95° C. for 5 seconds, an annealing step at 56° C. for 30 seconds, an extension step at 72° C. for 5 seconds, and a step of collecting fluorescent intensity. After the reaction was complete, the PCR products were run on a 1.2% w/v agarose gel (100V) for 30 minutes. Products exhibiting a single band with an expected size were sent to a company for sequencing. The sequencing results showed the presence of partial sequences of the sulfite reductase β subunit-encoding gene (DsrB) of cable bacteria (Candidatus Electronema), indicating that the primer pair of the present invention was highly specific for the DsrB gene of cable bacteria (Candidatus Electronema). The electrophoresis result was as shown in FIG. 1 and the PCR product had a sequence of SEQ ID NO: 3.

3) Preparing Positive Plasmid Standards

The PCR product was joined using a pEASY-T1 Simple Cloning Kit (TransGen Biotech), and transformed into Trans1-T1 phage resistant chemically competent cells. Correct white positive clones were identified by colony PCR and expanded. Positive plasmid was extracted using a plasmid mini kit, and named as pEASY-T1-DsrB. The obtained positive plasmid samples were sent to company for sequencing, wherein the positive plasmid pEASY-T1-DsrB verified to contain the positive fragment (comprising a sequence of SEQ ID NO: 3) was diluted to a plasmid concentration of 10¹⁰ copies/μL.

### Embodiment 2

Establishing Fluorescence Quantitative PCR Standard Curve

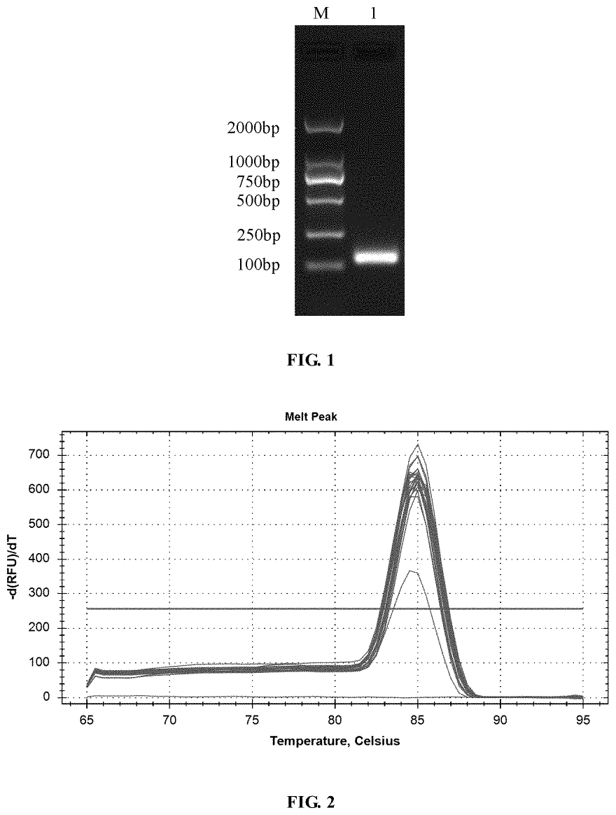

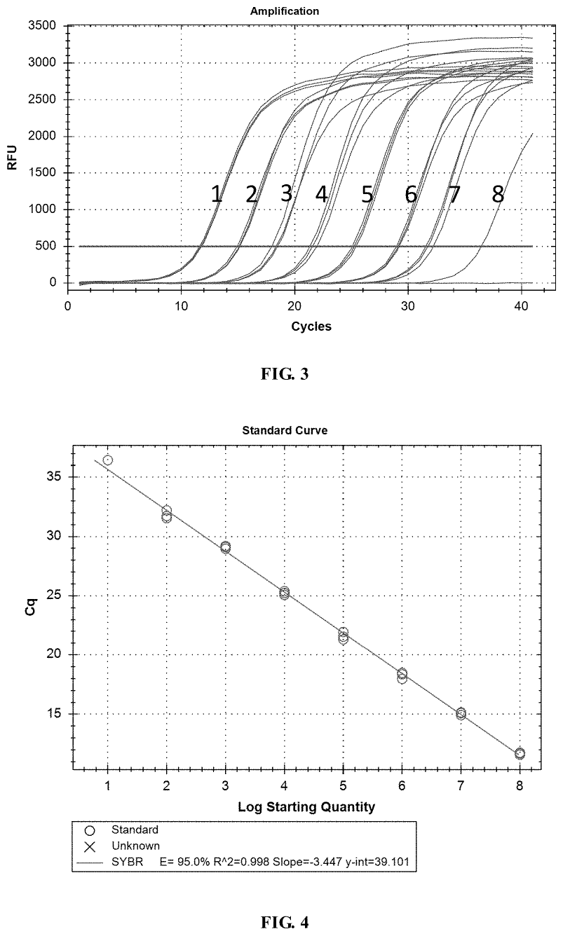

The positive plasmid obtained in Embodiment 1 was diluted to eight serial dilutions ranging from 10¹ to 10¹⁰ copies/μL, which were used as templates for fluorescence quantitative PCR, wherein 1 represents 10⁸ copies/μL, 2 represents 10⁷ copies/μL, 3 represents 10⁶ copies/μL, 4 represents 10⁵ copies/μL, 5 represents 10⁴ copies/μL, 6 represents 10³ copies/μL, 7 represents 10² copies/μL, and 8 represents 10¹ copies/μL. Dissociation curves and kinetic curves were plotted, wherein the dissociation curves were as shown in FIG. 2 and the kinetic curves were as shown in FIG. 3. In a second trial, The positive plasmid obtained in Embodiment 1 was diluted to eight serial dilutions ranging from 10¹ to 10¹⁰ copies/μL, which were used as templates for fluorescence quantitative PCR, wherein 1 represents 10¹ copies/μL, 2 represents 10² copies/μL, 3 represents 10³ copies/μL, 4 represents 10⁴ copies/μL, 5 represents 10⁵ copies/μL, 6 represents 10⁶ copies/μL, 7 represents 10⁷ copies/μL, and 8 represents 10⁸ copies/μL. CT values corresponding to each initial concentration were recorded and thereby a qPCR standard curve was plotted, as shown in FIG. 4.

The amplification reaction mixture comprises, per 25 μL of the amplification reaction mixture: 1 μL of a 10 μmol/L solution of the forward primer, 1 μL of a 10 μmol/L solution of the reverse primer, 12.5 μL of TB Green® Premix Ex Taq™ II, 1 μL of a solution of the positive plasmid, and 9.5 μL of ultrapure water.

The fluorescence quantitative PCR protocol comprised: (i) an initial denaturation step at 95° C. for 3 minutes, and (II) 40 cycles, wherein each of the cycles comprises a denaturation step at 95° C. for 5 seconds, an annealing step at 56° C. for 30 seconds, an extension step at 72° C. for 5 seconds, and a step of collecting fluorescent intensity.

The qPCR standard curve gave the following equation:

y=−3.447x+39.101, wherein E=95.0%, R²=0.998, x represents the common logarithm of plasmid initial concentration, and y represents the CT value.

As can be concluded from the equation, the CT values exhibited a linear relationship with the common logarithms of the initial concentrations with a R² value of 0.998, indicating an excellent correlation, and the amplification efficiency of the primers reached 95.0%.

As can be concluded from FIG. 2, the dissociation curves of different plasmid concentrations all peaked at a similar temperature, indicating a high specificity of the primers. As can be concluded from FIG. 3 and FIG. 4, the curves were parallel at the exponential phase (which indicates the amplification efficiency was similar for different concentrations), the CT values for different concentrations increased evenly, and the CT values exhibited a good linear relationship with the common logarithms of the initial concentrations.

As can be concluded from the results, the kit and methods of the present invention have high sensitivity, high accuracy, and good reproducibility, and allow detection with a linear range of 10¹-10⁸ copies/μL.

### Embodiment 3

Determining Specificity by Fluorescence Quantitative PCR

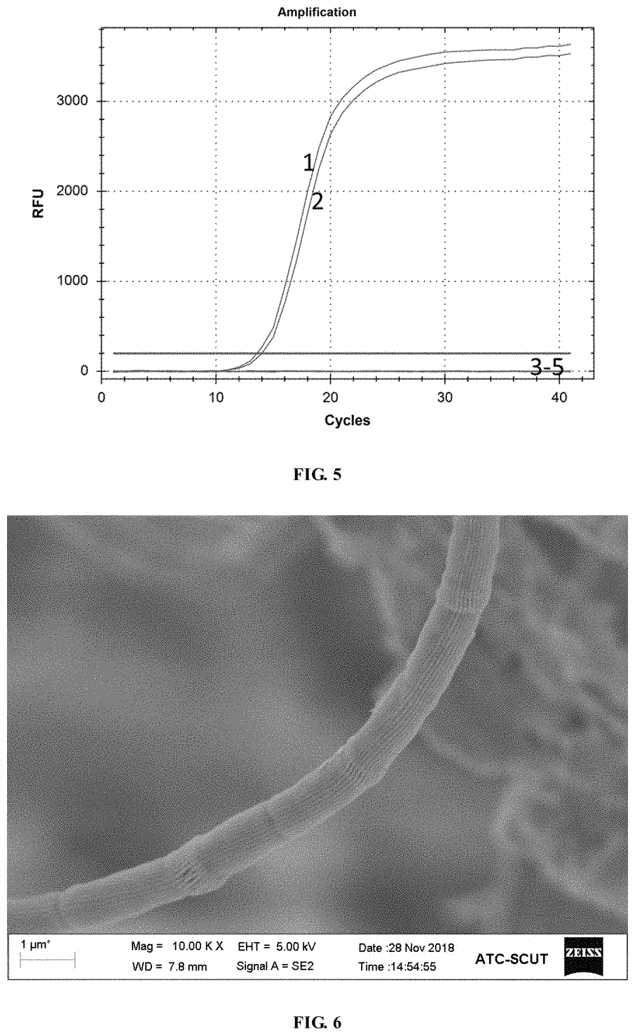

Fluorescence quantitative PCR was performed to test cable bacteria-rich samples (which were known to contain cable bacteria Candidatus Electronema), a Desulfobulbus spp. of the family Desulfobulbaceae, and E. coli. Results were as shown in FIG. 5, wherein 1 and 2 represent cable bacteria-rich positive samples, 3 represents a Desulfobulbus spp. of the family Desulfobulbaceae as a negative control, 4 represents E. coli as a negative control, and 5 represents water as a blank control. DNA extraction was performed using a soil DNA kit for the cable bacteria-rich samples, and using a conventional genomic DNA extraction method for Desulfobulbus spp. and E. coli. FIG. 6 is an SEM image of a cable bacteria-rich sample.

The amplification reaction mixture and the fluorescence quantitative PCR protocol were identical to those of Embodiment 2.

As can be seen from FIG. 5, fluorescent signals were observed for the cable bacteria enrichment culture, indicating the occurrence of amplification; for Desulfobulbus spp. and E. coli, no amplification occurred. It is thus suggested that the method of the present invention has a high specificity.

### Embodiment 4

Inspecting growth of the cable bacteria in the environmental samples by fluorescence quantitative PCR

Cable bacteria mediate LDET to generate electrical currents that link sulfide oxidation in deeper anoxic zones to oxygen reduction in surface oxic zones which are spatially segregated in sediments. In the presence of oxygen, the filamentous cable bacteria will grow vertically downward from the sediment surface to a size of 2-3 cm.

The experiment was performed on river sediments collected from an industrial zone in Pearl River basin (Ronggui, Shunde District, Foshan City). The sediments were sieved to remove large contaminations and mixed evenly, and then added into 100 mL beakers, each beaker containing about 80 mL of the sediments. The beakers along with the sediments were placed into a water tank, wherein tap water was added into the water tank until the water level was about 10 cm over the beakers. The water was aerated using a submersible pump to oxygen saturation. Having been respectively incubated on day 0, day 1, day 3, day 6, and day 9, the surface 2-cm of sediments in the beakers were collected, and three parallels were collected for each period, amounting to fifteen samples. A common soil DNA kit was used to extract total DNA from the samples.

1) 16S rRNA Gene Sequence Analysis

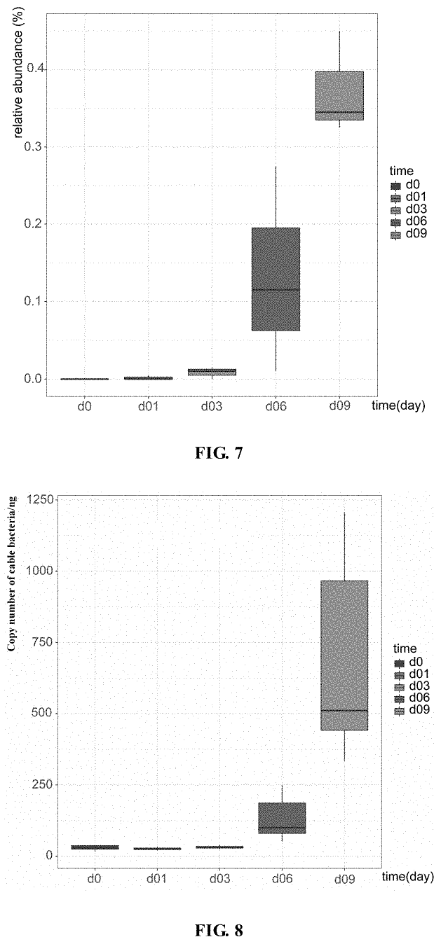

The DNA samples extracted from the fifteen environmental samples were subjected to 16S rRNA gene sequence of the V3-V4 region. The 16S rRNA gene sequence analysis (FIG. 7) showed that, the cable bacteria (Candidatus Electronema) exhibited a relative abundance of 0% at day 0, which did not change much at day 1 and day 3, but reached 0.1% at day 6 and 0.35% at day 9. Candidatus Electronema exhibited exponential growth since day 6.

2) Determining Abundance of Cable Bacteria in Environmental Samples by Fluorescence Quantitative PCR

The DNA samples extracted from the fifteen environmental samples were subjected to PCR and a standard curve was plotted, wherein the amplification reaction mixture and the fluorescence quantitative PCR protocol were identical to those of Embodiment 2. The copy number of DsrB gene of cable bacteria (Candidatus Electronema) in 1 ng DNA was calculated based on the standard curve and concentrations of the DNA samples, and thereby the copy number of Candidatus Electronema per 1 ng DNA was calculated. Results were as shown in FIG. 8. The copy number of Candidatus Electronema was 30 copies/ng DNA at day 0, did not change much at day 1 and day 3, but reached 130 copies/ng DNA at day 6 and 660 copies/ng DNA at day 9. Candidatus Electronema exhibited exponential growth since day 6.

As can be concluded from FIG. 7 and FIG. 8, the two methods, 16S rRNA gene sequence analysis and fluorescence quantitative PCR, gave consistent results on the abundance change and growth of Candidatus Electronema in sediments. However, the fluorescence quantitative PCR method exhibited a higher sensitivity and a lower minimum detection limit.

As can be concluded from the above embodiments, the kit and method of the present invention have high sensitivity, high accuracy, good reproducibility, and high specificity, and allow detection with a linear range of 10¹-10⁸ copies/μL.

It should be noted that, for those of ordinary skill in the art, without departing from the principle of the present invention, several improvements and modifications can be made, and these improvements and modifications should also fall within the protection scope of the present invention.

1. A primer set for detecting cable bacteria Candidatus Electronema, comprising:
a forward primer as shown in SEQ ID NO: 1, and
a reverse primer as shown in SEQ ID NO: 2.

2. A kit for detecting cable bacteria Candidatus Electronema, comprising a reagent necessary for detection, a positive control, and the primer set of claim 1, wherein the positive control is a recombinant plasmid DNA comprising a sequence of SEQ ID NO: 3.

3. A method for detecting cable bacteria Candidatus Electronema, comprising the following steps: extracting genomic DNA from a sediment sample, mixing the genomic DNA with the primer set of claim 1 and a reagent necessary for detection to obtain an amplification reaction mixture, performing fluorescence quantitative PCR, and determining a presence of the cable bacteria Candidatus Electronema in the sediment sample based on amplification curves.

4. The method of claim 3, wherein the step of determining the presence of the cable bacteria Candidatus Electronema in the sediment sample based on the amplification curves is performed according to the following standard: if the amplification curves comprise typical amplification curves and a cycle threshold value is below 35, then the cable bacteria Candidatus Electronema are present in the sediment sample; if the amplification curves do not comprise the typical amplification curves, then the cable bacteria Candidatus Electronema are not present in the sediment sample.

5. The method of claim 3, wherein the amplification reaction mixture comprises, per 25 μL of the amplification reaction mixture: 1 μL of a 10 μmol/L solution of the forward primer of claim 1, 1 μL of a 10 μmol/L solution of the reverse primer of claim 1, 12.5 μL of the reagent necessary for the detection, 1 μL of an extracted solution of the genomic DNA, and 9.5 μL of ultrapure water;
the step of performing the fluorescence quantitative PCR comprises: (i) an initial denaturation step at 95° C. for 3 minutes, and (II) 40 cycles, wherein each of the 40 cycles comprises a denaturation step at 95° C. for 5 seconds, an annealing step at 56° C. for 30 seconds, an extension step at 72° C. for 5 seconds, and a step of collecting a fluorescent intensity.

6. A method for quantitating an abundance of cable bacteria Candidates Electronema, comprising the following steps:
(1) producing a recombinant plasmid by joining a sequence of SEQ ID NO: 3 with a plasmid, serially diluting the recombinant plasmid to obtain template solutions of a plurality of initial concentrations, mixing each of the template solutions with the primer set of claim 1 and a reagent necessary for detection to obtain a first amplification reaction mixture, and performing a first fluorescence quantitative PCR on the first amplification reaction mixture;
(2) recording CT values corresponding to the initial concentrations, and obtaining a standard curve by plotting the CT values against common logarithms of the initial concentrations, wherein the CT values exhibit a linear relationship with the common logarithms of the initial concentrations;
(3) extracting genomic DNA from a sediment sample, mixing the genomic DNA with the primer set and the reagent used in step (1) to obtain a second amplification reaction mixture, performing a second fluorescence quantitative PCR on the second amplification reaction mixture, recording a sample CT value, and substituting the sample CT value into the standard curve to obtain the abundance of the cable bacteria Candidatus Electronema in the sediment sample.

7. The method of claim 6,
wherein in step (1), the first amplification reaction mixture comprises, per 25 μL of the first amplification reaction mixture: 1 μL of a 10 μmol/L solution of the forward primer, 1 μL of a 10 μmol/L solution of the reverse primer, 12.5 μL of the reagent, 1 μL of the recombinant plasmid, and 9.5 μL of ultrapure water;
in step (3), the second amplification reaction mixture comprises, per 25 μL of the second amplification reaction mixture: 1 μL of the 10 μmol/L solution of the forward primer, 1 μL of the 10 μmol/L solution of the reverse primer, 12.5 μL of the reagent, 1 μL of an extracted solution of the genomic DNA, and 9.5 μL of the ultrapure water;
in step (1) and step (3), the step of performing the first fluorescence quantitative PCR and the second fluorescence quantitative PCR each comprises: (i) an initial denaturation step at 95° C. for 3 minutes, and (II) 40 cycles, wherein each of the 40 cycles comprises a denaturation step at 95° C. for 5 seconds, an annealing step at 56° C. for 30 seconds, an extension step at 72° C. for 5 seconds, and a step of collecting a fluorescent intensity.
